(19) United States
(12) Patent Application Publication
(10) Pub. No.: US 2024/0342478 A1
(43) Pub. Date: Oct. 17, 2024

(54) Phrenic Nerve Pacing

(71) Applicants: Rodney Paul Horton, Austin, TX (US); Francisco Javier Otero, Austin, TX (US)

(72) Inventors: Rodney Paul Horton, Austin, TX (US); Francisco Javier Otero, Austin, TX (US)

(21) Appl. No.: 18/635,662

(22) Filed: Apr. 15, 2024

Related U.S. Application Data
(60) Provisional application No. 63/495,889, filed on Apr. 13, 2023.

(51) Int. Cl. A61N 1/36

(52) U.S. Cl. CPC A61N 1/3611; A61N 1/36139

(57) ABSTRACT

A method of phrenic nerve pacing. The method comprises positioning an electrical sensor within vasculature of a subject. The method comprises directly sensing, via the electrical sensor during spontaneous respiration of the subject, efferent activity of a phrenic nerve of the subject.

### CROSS REFERENCE TO RELATED APPLICATION

This application is based upon and claims benefit of priority to U.S. Provisional Application No. 63/495,889, filed Apr. 13, 2023, and hereby incorporated by reference herein in its entirety.

### FIELD

Disclosed herein is a system and method that may be used to perform phrenic nerve pacing.

### BACKGROUND

The left and right phrenic nerves originate in the neck and travel downward into the chest to the diaphragm. The left and right phrenic nerves provide motor function to the diaphragm, with the left phrenic nerve innervating the left diaphragmatic dome and the right phrenic nerve innervating the right diaphragmatic dome. Phrenic nerve injury may be a common condition that occurs as a consequence of heart procedures and may occur spontaneously in the elderly. Injury or damage to the phrenic nerve may lead to diaphragm weakness or paralysis and affects the lungs' ability to exchange air. Unilateral injury, in which either the left or right phrenic nerve is injured, may cause weakness or paralysis in one side of the diaphragm. Bilateral injury, in which the left and right phrenic nerves are both injured, may cause weakness or paralysis in both sides of the diaphragm and may cause spontaneous sudden death. Although bilateral injury is not compatible with life, and therefore, is generally not recognized, unilateral injury may be treated with phrenic nerve pacing, which is also known as diaphragmatic pacing.

Phrenic nerve pacing involves electrical stimulation of the injured phrenic nerve to cause diaphragm contractions. Technology exists that uses electrical stimulation of the phrenic nerve by placing a stimulating electrode in a vein adjacent to the thoracic level of the phrenic nerve. While this technique may have some advantages over direct stimulation of the diaphragmatic muscles, this technique poses some disadvantages because the technique requires the site of the phrenic nerve damage to be proximal (more cranial) to the site of the pacing.

Existing phrenic nerve pacing technology is used in somewhat limited applications. For example, the trans-thoracic impedance sensing method is used to detect apnea and pace during sleep cycles in patients suffering from central sleep apnea. The primary reason for such limited application may be due to the inability to replicate brain stem function. Brain stem function senses blood pH, CO₂, and O₂ levels and controls respiratory regulation accordingly. Because the brain stem senses and uses blood pH, CO₂, and O₂ levels, creating and using electronic sensing technology to replicate the brain stem's control of respiratory regulation may be complex and likely beyond the ability of current technology. Therefore, current phrenic nerve pacers may send diaphragm signals differently from natural phrenic nerve impulses which are primarily controlled by brain stem output.

Thus, there is a need for a phrenic nerve pacing method and system that addresses one or more of the deficiencies of existing phrenic nerve pacing methods and systems. For example, the inventors have identified a need for a phrenic nerve pacing method and system that may directly detect brain stem output to regulate phrenic nerve pacers to replicate natural phrenic nerve activity.

### SUMMARY

Described herein, in various aspects, is a method of phrenic nerve pacing. The method may comprise positioning an electrical sensor within vasculature of a subject and directly sensing, via the electrical sensor during spontaneous respiration of the subject, efferent activity of a phrenic nerve of the subject.

In another aspect, a system for phrenic nerve pacing includes an electrical sensor, a stimulating electrode, and a processor. The electrical sensor may be configured to be positioned within vasculature of the subject and to sense efferent activity of one of the left phrenic nerve or the right phrenic nerve of the subject. The stimulating electrode may be configured to be positioned within vasculature of the subject and to electrically stimulate the other of the left phrenic nerve of the right phrenic nerve of the subject. The processor may be in communication with the electrical sensor and the stimulating electrode. The processor may be configured to receive signals from the electrical sensor corresponding to the sensed efferent activity of one of the left phrenic nerve or the right phrenic nerve of the subject. The processor may be further configured to cause the stimulating electrode to apply electrical stimulation to the other of the left phrenic nerve or the right phrenic nerve of the subject. The electrical stimulation may be configured to synchronize movement of left and right sides of the diaphragm of the subject.

### DESCRIPTION OF THE DRAWINGS

FIGS. 1-2 are block diagrams showing elements of an exemplary phrenic nerve pacing system as disclosed herein.

FIG. 3 is a diagram of a vasculature system showing exemplary approximate locations for an electrical sensor.

FIG. 4 is a diagram of cervical nerves in a right side of a neck showing exemplary approximate locations for the electrical sensor.

FIG. 5 is a diagram of cervical nerves in a left side of a neck showing exemplary approximate locations for the electrical sensor.

FIG. 6 is a second diagram of a vasculature system showing exemplary approximate locations for the electrical sensor.

FIG. 7 is a block diagram of an operating environment comprising a computing device as disclosed herein for controlling the system of FIG. 1.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” can optionally include plural referents unless the context clearly dictates otherwise. For example, unless the context dictates otherwise, use of the term “a signal” can be interpreted as “at least one signal” and can represent disclosure of embodiments in which only a single such signal is provided, as well as embodiments in which a plurality of such signals are provided.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “approximately,” “substantially,” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where said event or circumstance occurs and instances where it does not.

As used herein, the term “subject” refers to both human and animal subjects.

The following description supplies specific details in order to provide a thorough understanding. Nevertheless, the skilled artisan would understand that the system and associated methods of using the system can be implemented and used without employing these specific details. Indeed, the system and associated methods can be placed into practice by modifying the illustrated apparatus and associated methods and can be used in conjunction with any other apparatus and techniques conventionally used in the industry.

### Introduction

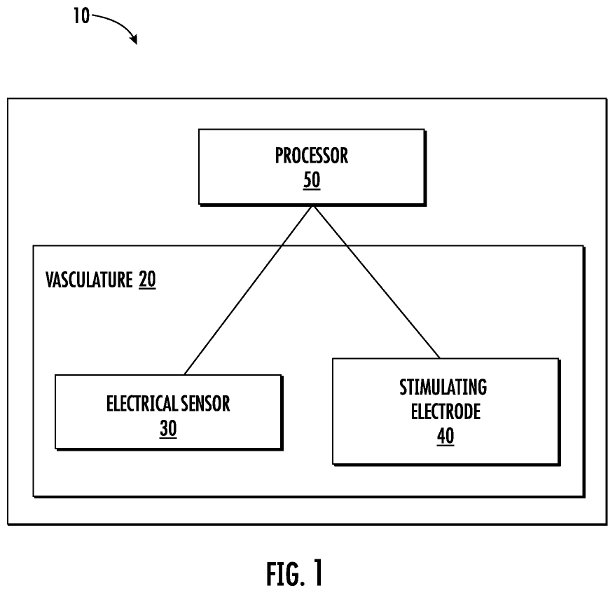

Described herein, with reference to FIG. 1, is a system 10 for phrenic nerve pacing. The system 10 may detect brain stem output of a subject, via efferent activity of a non-injured left or right phrenic nerve, to regulate phrenic nerve pacers according to natural phrenic nerve activity. An electrical sensor 30 may sense the efferent activity of an uninjured, left or right phrenic nerve. A processor 50 may receive the sensed efferent activity from the electrical sensor 30 and cause a stimulating electrode 40 to electrically stimulate the other one of the left or right phrenic nerve (which can optionally be injured) according to the received sensed efferent activity. The stimulating electrode 40 may be a component of a phrenic pacing device or cardiac pacing device known in the art. The system 10 may be configured to operate with a phrenic pacing device or cardiac pacing device to treat defects in respiration.

### Exemplary Embodiments

In exemplary aspects, as shown in FIG. 1, the system 10 may comprise an electrical sensor 30 (optionally, a plurality of such sensors). The electrical sensor 30 may be configured to be positioned within the vasculature 20 of a subject. Further, the electrical sensor 30 may be configured to sense efferent activity of the left phrenic nerve or the right phrenic nerve. In one example, the subject may have experienced a phrenic nerve injury. In this example, the subject may have injured either the left phrenic nerve or the right phrenic nerve causing diaphragm weakness or paralysis in the left side of the diaphragm or right side of the diaphragm, respectively. The electrical sensor 30 may be configured to sense efferent activity of the uninjured phrenic nerve. For example, if the subject injured the left phrenic nerve, the electrical sensor 30 may be configured to sense efferent activity of the right phrenic nerve. If the subject injured the right phrenic nerve, the electrical sensor 30 may be configured to sense efferent activity of the left phrenic nerve.

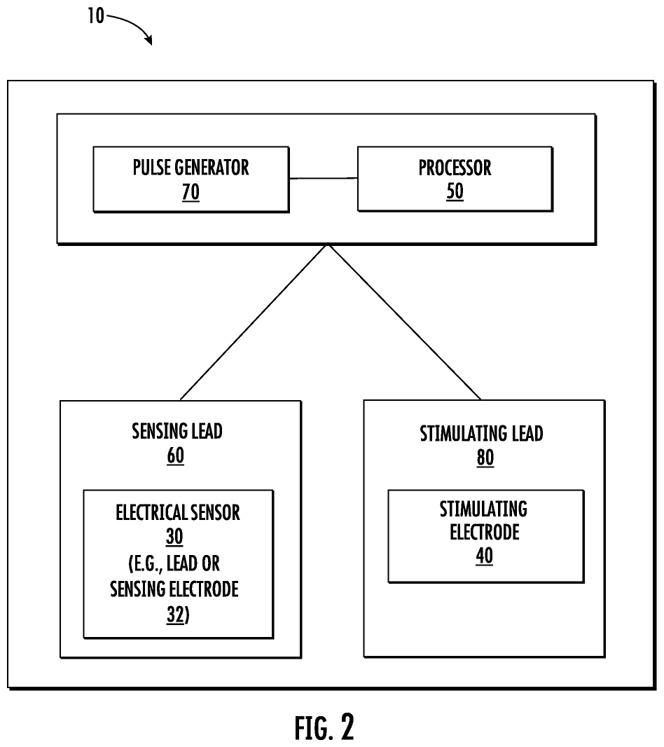

In one aspect, the electrical sensor 30 may comprise or be a portion of a sensing electrode 32. The sensing electrode 32 may sense the efferent electrical activity of the uninjured phrenic nerve. For example, the sensing electrode 32 may measure a voltage (and a change in voltage over time) associated with the electrical activity of the uninjured phrenic nerve. In further aspects, with reference to FIG. 2, the electrical sensor 30 (e.g., lead or sensing electrode 32) may be a component of a sensing lead 60 as is known in the art. The sensing lead 60 can have a flexible, deformable body. In some aspects, it is contemplated that the sensing lead 60 can be an intravascular recording lead as is known in the art. For example, in these aspects, the electrical sensor 30 can comprise a first electrode (e.g., a tip electrode) and/or a second electrode (e.g., a ring electrode) of the intravascular recording lead, and the first and/or second electrodes can be used to record nerve activity and/or to provide intravascular pacing or recording. Optionally, the intravascular recording lead can include a suturing hole or other attachment mechanism that permits secure placement of the intravascular recording lead over a target location such as further disclosed herein. In other aspects, it is contemplated that the sensing lead 60 can comprise a probe or catheter structure. The sensing lead 60 may be used to position the electrical sensor 30 (e.g., sensing electrode 32) in the subject's vasculature 20 at a location where the electrical sensor 30 may sense the efferent activity of the left or right phrenic nerve. The sensing lead 60 may be connected or coupled to an implanted medical device (IMD). In use, it is contemplated that the electrical sensor can be configured to sense and transmit efferent electrical signals (e.g., voltage and/or action potential over time) provided by the phrenic nerve.

In one aspect, to sense the efferent activity of the uninjured phrenic nerve, the electrical sensor 30 may be positioned within the vasculature 20 in a cranial direction (toward the head) with respect to the heart of the subject.

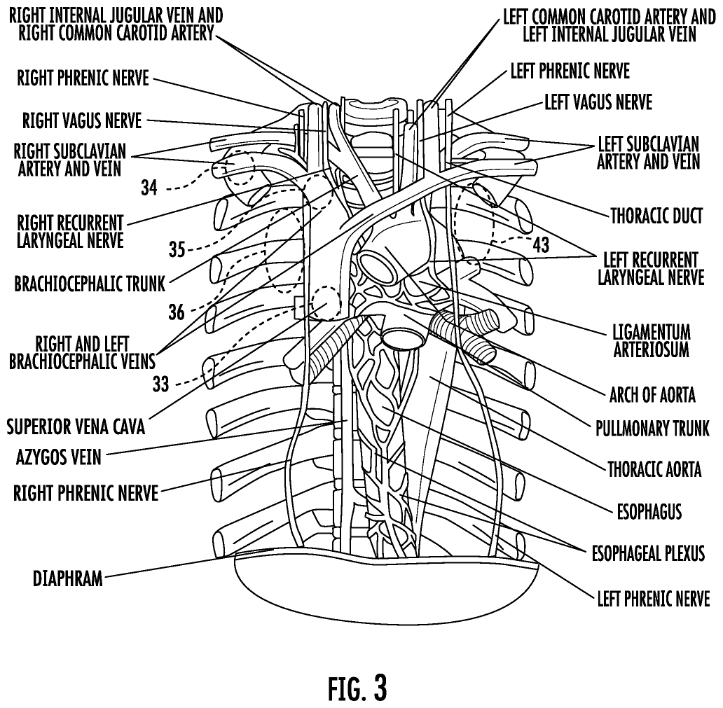

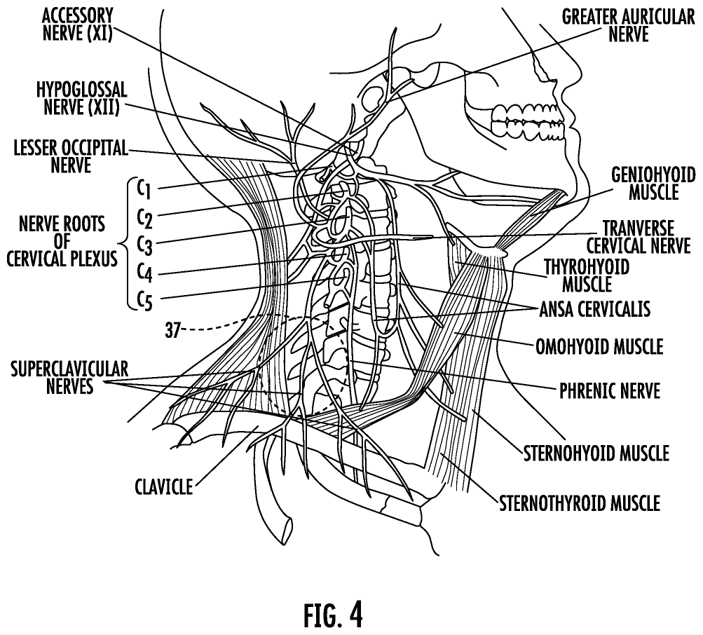

The electrical sensor 30 may be positioned in various locations within the vasculature 20 to sense the efferent activity of an uninjured right phrenic nerve of a subject. In a first example and with reference to FIG. 3, the electrical sensor 30 may be positioned within the subject's superior vena cava at approximate location 33 to sense the efferent activity of the right phrenic nerve of the subject. In a second example, the electrical sensor 30 may be positioned within the subject's right subclavian vein at approximate location 34 to sense the efferent activity of the right phrenic nerve of the subject. In a third example, the electrical sensor 30 may be positioned within a junction between the subject's superior vena cava and the right subclavian vein at approximate location 35 to sense the efferent activity of the right phrenic nerve of the subject. In a fourth example, the electrical sensor 30 may be positioned within the thoracic cavity adjacent to the right phrenic nerve at approximate location 36 to sense the efferent activity of the right phrenic nerve of the subject. In a fifth example and with reference to FIG. 4, the electrical sensor 30 may be positioned at an extravascular location along a nerve course in the subject's right side of neck at approximate location 37 to sense the efferent activity of the right phrenic nerve of the subject. Although each of the above-referenced locations is described as a separate example, it is contemplated that in some exemplary aspects, respective electrical sensors 30 of a plurality of electrical sensors can be positioned at different locations (e.g., two or more of the superior vena cava, the right subclavian vein, the junction between the superior vena cava and the right subclavian vein, the thoracic cavity adjacent to the right phrenic nerve, or an extravascular location along the nerve course in the right side of the neck) to permit sensing of efferent activity of the right phrenic nerve at multiple locations.

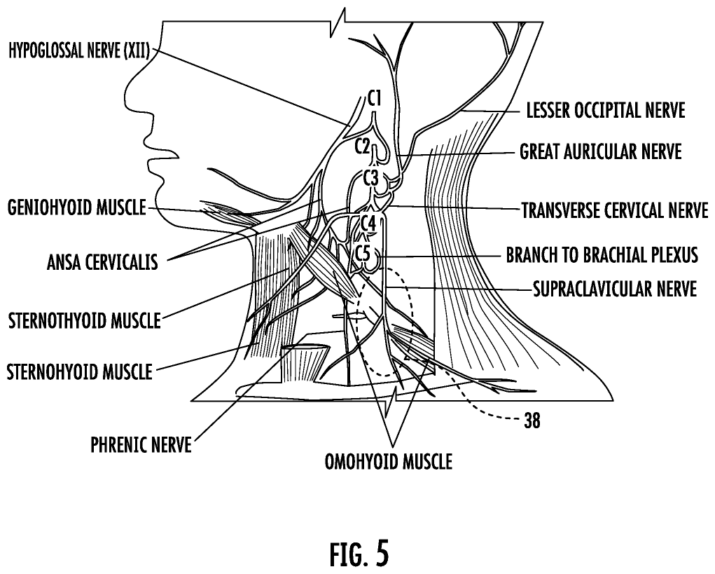

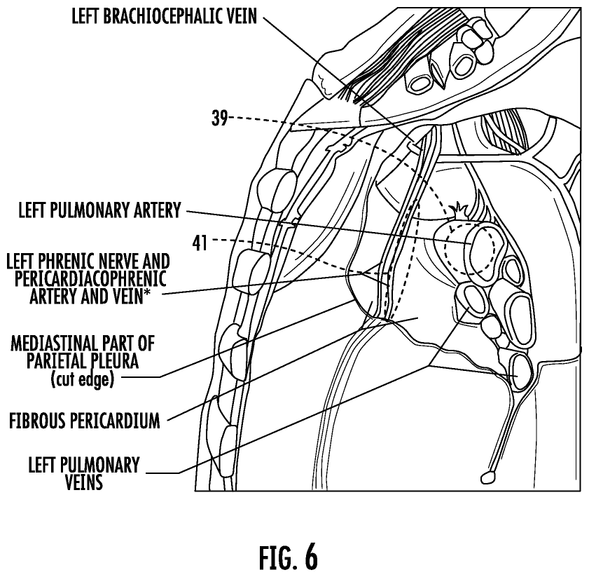

The electrical sensor 30 may be positioned in various locations within the vasculature 20 to sense the efferent activity of an uninjured left phrenic nerve of a subject. In a first example, and with reference to FIG. 5, the electrical sensor 30 may be positioned at an extravascular location along a nerve course in the subject's left side of the neck at approximate location 38 to sense the efferent activity of the left phrenic nerve of the subject. In a second example, and with reference to FIG. 6, the electrical sensor 30 may be positioned within the subject's left pulmonary artery at approximate location 39 to sense the efferent activity of the left phrenic nerve of the subject. In a third example, the electrical sensor 30 may be positioned within the subject's left pericardiocophrenic vein at approximate location 41 to sense the efferent activity of the left phrenic nerve of the subject. In a fourth example, and with reference to FIG. 3, the electrical sensor 30 may be positioned within the subject's left thoracic cavity at approximate location 43 to sense the efferent activity of the left phrenic nerve of the subject. Although each of the above-referenced locations is described as a separate example, it is contemplated that in some exemplary aspects, respective electrical sensors 30 of a plurality of electrical sensors can be positioned at different locations (e.g., two or more of an extravascular location along the nerve course in the left side of the neck, the left pulmonary artery, the left pericardiocophrenic vein, or the left thoracic cavity) to permit sensing of efferent activity of the left phrenic nerve at multiple locations.

With reference back to FIG. 1, the system 10 may comprise at least one stimulating electrode 40 (optionally, a plurality of stimulating electrodes). Each stimulating electrode 40 may be positioned within vasculature 20 of the subject. In one aspect, each stimulating electrode 40 may be a component of a phrenic nerve pacing device or cardiac pacing device as is known in the art. The pacing device may be an implantable medical device configured to provide phrenic nerve stimulation therapy. Additionally, or alternatively, it is contemplated that the stimulating electrode 40 can be a component of, or be coupled to or associated with, a stimulation lead system 80 as is known in the art. Optionally, the stimulation lead system 80 can include at least one intravascular pacing/stimulation lead as is known in the art. Optionally, the lead may be a transvenous lead which passes from an IMD through venous vasculature to enter a vein. The stimulation lead may be configured to be implanted in a vein proximate to the phrenic nerve it is configured to stimulate. For example the stimulation lead may be implanted within the cardiophrenic vein or pericardiophrenic vein. In other aspects, it is contemplated that the stimulation lead can comprise at least one probe or catheter structure. The stimulation lead may include multiple stimulating electrodes 40 along the length of the lead.

The at least one stimulating electrode 40 may be configured to electrically stimulate the left phrenic nerve or the right phrenic nerve. In the example involving a subject with an injured or damaged left phrenic nerve or right phrenic nerve, the stimulating electrode 40 may be configured to electrically stimulate the injured or damaged phrenic nerve. For example, if the subject injured the left phrenic nerve and has a healthy right phrenic nerve, the stimulating electrode 40 may be configured to electrically stimulate the left phrenic nerve while the electrical sensor 30 detects and/or measures electrical activity within the right phrenic nerve. If the subject injured the right phrenic nerve and has a healthy left phrenic nerve, the stimulating electrode 40 may be configured to stimulate the right phrenic nerve while the electrical sensor 30 detects and/or measures electrical activity within the left phrenic nerve. The stimulating electrode 40 may stimulate the injured phrenic nerve to provide motor function to the corresponding side of the diaphragm.

As shown in FIG. 1 and FIG. 2, the system 10 may comprise a processor 50. The processor 50 may be a component of an IMD. The processor 50 may be in communication with the electrical sensor 30 and the stimulating electrode 40. Optionally, the processor 50 may communicate with a pulse generator 70. The processor 50 may be configured to receive signals from the electrical sensor 30 corresponding to the sensed efferent activity of the uninjured phrenic nerve. The processor 50 may also be configured to cause the stimulating electrode 40 to apply electrical stimulation to the damaged phrenic nerve of the subject. The processor 50 may cause the stimulating electrode to apply electrical stimulation via the pulse generator 70. The electrical stimulation may be configured to synchronize movement of the left and right sides of the diaphragm of the subject. The processor 50 may allow the stimulating electrode 40 to control the damaged phrenic nerve, and therefore, the movement of the corresponding side of the diaphragm, according to the efferent activity of the uninjured or undamaged phrenic nerve on the other side of the diaphragm. For example, for each period of efferent activity detected within the uninjured/undamaged phrenic nerve by the electrical sensor(s), the stimulating electrode(s) can be configured to provide stimulation that produces corresponding (or substantially corresponding) efferent activity. The processor 50 thereby regulates the stimulating electrode 40 to replicate natural phrenic nerve activity, i.e. the nerve activity of the uninjured phrenic nerve, allowing the left and right sides of the diaphragm to be substantially synchronized despite an injured or damaged phrenic nerve.

### Method of Use

A method may comprise positioning an electrical sensor 30 within vasculature 20 of a subject. The electrical sensor 30 may directly sense efferent activity of a phrenic nerve of the subject during spontaneous respiration (e.g., natural respiration produced in response to movement of the respiratory muscles) of the subject. Optionally, direct sensing of efferent activity can occur when the electric sensor is in direct electrical communication with (optionally, direct electrical contact with) the phrenic nerve. Thus, the signals received by the electrical sensor are indicative of the actual efferent activity of the nerve.

In some aspects, the electrical sensor 30 may comprise a sensing electrode 32. In some further aspects, the sensing electrode 32 may be a component of a sensing lead 60 as further disclosed herein.

In some aspects, the electrical sensor 30 may be spaced from the heart of the subject in a cranial direction.

In some aspects, the electrical sensor 30 may directly sense efferent activity of the right phrenic nerve of the subject. In a further aspect, the electrical sensor 30 may be positioned within the superior vena cava, within the right subclavian vein, or at a junction between the superior vena cava and the right subclavian vein of the subject. In another aspect, the electrical sensor 30 may be positioned within a thoracic cavity adjacent to the phrenic nerve or at an extravascular location along a nerve course in a neck of the subject. Optionally, direct sensing of efferent activity can occur when the electric sensor is in direct electrical communication with (optionally, direct electrical contact with) the phrenic nerve. Thus, the signals received by the electrical sensor are indicative of the actual efferent activity of the nerve.

In some aspects, the method may further comprise stimulating the left phrenic nerve of the subject based on the sensed efferent activity of the right phrenic nerve. In a further aspect, the left phrenic nerve may be electrically stimulated by a stimulating electrode 40 positioned within vasculature 20 of the subject. The left phrenic nerve of the subject may be stimulated in a pattern corresponding to a pattern of the efferent activity of the right phrenic nerve of the subject.

In some aspects, the diaphragm of the subject may be defective. For example, when the left diaphragm is defective, the electrical stimulation of the left phrenic nerve may effect movement of the left side of the diaphragm in a pattern that corresponds to a pattern (e.g., natural pattern) of movement of a right side (optionally, healthy right side) of the diaphragm of the subject. Similarly, when the right diaphragm is defective, the electrical stimulation of the right phrenic nerve may effect movement of the right side of the diaphragm in a pattern that corresponds to a pattern (e.g., natural pattern) of movement of a left side (optionally, healthy left side) of the diaphragm of the subject.

In some aspects, the electrical sensor 30 directly senses efferent activity of the left phrenic nerve of the subject. In these aspects, the right phrenic nerve of the subject may be electrically stimulated based on the sensed efferent activity of the left phrenic nerve. In a further aspect, the electrical sensor 30 may be positioned within an extravascular location along a left side of a neck, within a left pulmonary artery, within a left pericardiocophrenic vein, or a left thoracic cavity in the subject.

In some aspects, the right phrenic nerve may be electrically stimulated by a stimulating electrode 40 positioned within the vasculature 20 of the subject. In a further aspect, the right phrenic nerve of the subject may be stimulated in a pattern corresponding to a pattern of the efferent activity of the left phrenic nerve of the subject.

In some aspects, the right side of the diaphragm of the subject may be defective. The electrical stimulation of the right phrenic nerve may affect movement of the right side of the diaphragm in a pattern that corresponds to a pattern of movement of a left side of the diaphragm of the subject.

### Computing Device

The system 10 may comprise at least one computing device for controlling operation of the system 10. For example, one or more computing devices can control a plurality of operations, including: receiving a signal from the electrical sensor 30 corresponding to the sensed efferent activity of the uninjured phrenic nerve (either the left or right phrenic nerve); and causing the stimulating electrode 40 to apply electrical stimulation to the injured phrenic nerve (the other of the left or right phrenic nerve). In some optional aspects, a single computing device controls a plurality of such operations. For example, it is contemplated that a single computing device can be configured to both receive the signal from the electrical sensor 30 and apply electrical stimulation through the stimulating electrode 40. In some aspects, the system 10 can comprise a plurality of computing devices that operate in coordination. For example, a first computing device (by way of the processor 50) may receive the signal from the electrical sensor 30 corresponding to the sensed efferent activity of the uninjured phrenic nerve (either the left or right phrenic nerve). Still another computing device may cause the stimulating electrode 40 to apply electrical stimulation to the injured phrenic nerve (the other of the left or right phrenic nerve). Each of said computing devices can optionally be embodied in accordance with the computing device 1001 as further disclosed herein.

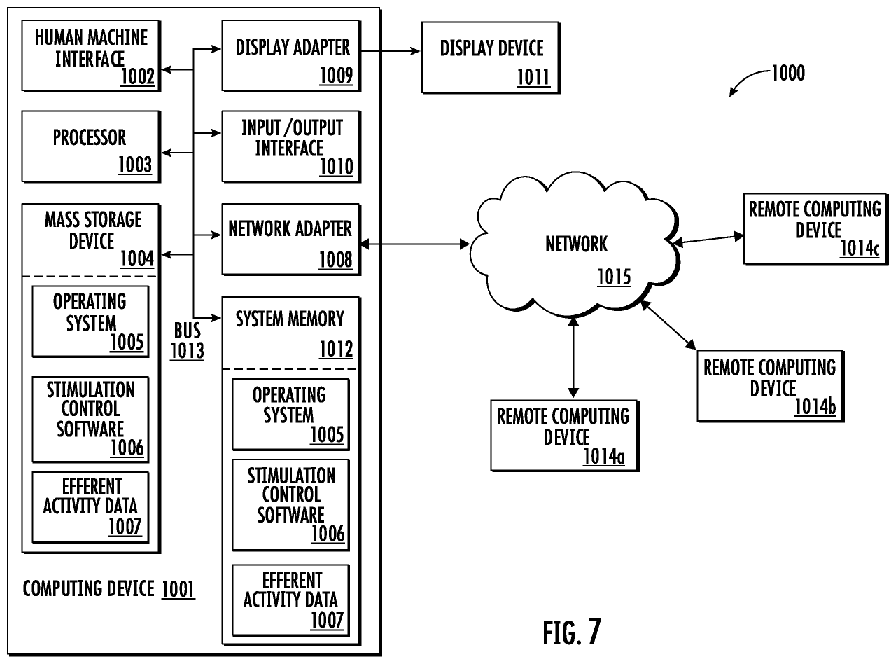

FIG. 7 shows an exemplary operating environment 1000 including an exemplary configuration of a computing device 1001 for use with the system 10 disclosed herein.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as phrenic nerve efferent activity data 1007 and/or program modules such as operating system 1005 and phrenic nerve stimulation control software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and phrenic nerve stimulation control software 1006 may be stored on the mass storage device 1004. One or more of the operating system 1005 and stimulation control software 1006 (or some combination thereof) may comprise program modules and the stimulation control software 1006. The efferent activity data 1007 may also be stored on the mass storage device 1004. The efferent activity data 1007 may be stored in any of one or more databases known in the art. The databases may be centralized or distributed across multiple locations within the network 1015.

A user may enter commands and information into the computing device 1001 using an input device. Such input devices comprise, but are not limited to, a joystick, a touchscreen display, a keyboard, a pointing device (e.g., a computer mouse, remote control), a microphone, a scanner, tactile input devices such as gloves, and other body coverings, motion sensor, speech recognition, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. The remote computing devices 1014a,b,c, can perform respective operations of the system. For example, one remote computing device 1014a can be in communication with the electrical sensor 30. One remote computing device 1014b can be in communication with and control the stimulating electrode 40. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### Exemplary Embodiments of the Disclosed Methods and Systems

The methods and systems disclosed herein may incorporate commercially available diaphragmatic/phrenic nerve stimulation devices or diaphragm pacing systems. For example, the methods and systems disclosed herein may utilize all or some of the hardware components of the phrenic nerve stimulation systems disclosed in the following patents, each of which is fully incorporated by reference herein in its entirety: U.S. Pat. Nos. 8,233,987, 8,244,359, 8,433,412, 9,295,846, 9,468,755, 9,744,349, 9,999,768, 10,406,366, 10,507,322, 10,518,090, each of which is assigned to ZOLL RESPICARDIA, INC. For example, it is contemplated that the methods and systems disclosed herein may utilize an implanted medical device (IMD) implanted in a patient's chest for sensing efferent activity of a phrenic nerve and/or stimulating a phrenic nerve of the patient. In this example, a transvenous lead can pass from the IMD through venous vasculature to enter a selected location of the venous vasculature for directly sensing efferent activity of the phrenic nerve and/or electrically stimulating the phrenic nerve. Electrical stimulation pulses supplied to a stimulation electrode on the lead can interact with the phrenic nerve to stimulate it and thus activate the diaphragm. In use, it is contemplated that the stimulation electrode can lie far enough away from the heart of the patient to avoid stimulating the heart. Optionally, only one branch of the phrenic nerve is stimulated and the other side of the nerve is under normal physiologic control.

As can be understood, the phrenic nerve stimulation system can have an implanted portion and an external programmer portion. The IMD can receive data indicative of the detected efferent activity of the phrenic nerve and/or provide stimulation pulses to the stimulation electrode. A companion indifferent electrode may be used to sink or source the stimulation current generated in analog circuits. When the therapy is invoked by being turned on by the programmer or in response to detecting a threshold or other parameter based on the direct detection of efferent activity of the phrenic nerve as disclosed herein, logic can command the stimulation of the phrenic nerve via the stimulation electrode (for example, at a time after the beginning of the inspiration phase). Optionally, the stimulation can begin after the onset of exhalation. However, it should be understood that there is some flexibility in onset of stimulation. Optionally, the logic can command stimulation at higher amplitudes of energy levels as the stimulation progresses. It may also be desirable to have stimulation ramp up and ramp down during the therapy. It may prove desirable to stimulate episodically. The therapy may be best administered to every other breath or in a random pattern.

The duration of the stimulation is under the control of the logic. Optionally, the therapy can be dispensed with a fixed duration of pulses based on the detected efferent activity of the phrenic nerve. However, it should be clear that other strategies for setting the duration of stimulation are within the scope of the disclosed systems and methods. Optionally, the disclosed system can include activity sensors, and the system may deliver therapy at times of relative inactivity (resting or sleeping). Activity sensors may also help in the detection and rejection of artifacts. An accelerometer, such as those used in cardiac pacing, is an exemplary activity sensor.

In some exemplary aspects, the disclosed methods can be performed using an implantable phrenic nerve stimulator, such as those that are indicated for the treatment of central sleep apnea (CSA) in adult patients. As one example, it is contemplated that the disclosed methods can be performed using a REMEDE implantable phrenic nerve stimulation system (ZOLL RESPICARDIA, INC.) that has been configured for use as disclosed herein.

The lead(s) disclosed in the above-discussed phrenic nerve stimulation systems may include, or be modified to include, one or more of the electrical sensors 30 described herein to directly sense efferent activity of a phrenic nerve of a subject. These lead(s) may be used to position at least one electrical sensor 30 at at least one of the locations described herein to sense the efferent activity. These lead(s) may include, or be modified to include, one or more of the stimulating electrodes 40 described herein to electrically stimulate a phrenic nerve of a subject. These lead(s) may be used to position at least one stimulating electrode 40 at at least one of the locations described herein to electrically stimulate a phrenic nerve.

Further, the sensing lead(s) disclosed in the above-discussed phrenic nerve stimulation systems may be used to position at least one sensor within vasculature of a subject. The sensing lead(s) disclosed in the above-discussed phrenic nerve stimulation systems may be used to sense efferent activity of a phrenic nerve of a subject. The electrical stimulation lead(s) disclosed in the above-discussed phrenic nerve stimulation systems may be used to position at least one electrode within vasculature of a subject. The electrical stimulation lead(s) disclosed in the above-discussed phrenic nerve stimulation systems may be used to electrically stimulate a phrenic nerve.

Moreover, the IMD(s) disclosed in the above-discussed phrenic nerve stimulation systems may include the processor 50 described herein. The electrical sensor(s) and the stimulating electrode(s) may communicate with the processor 50 of the IMD. Further, the sensor(s) and the electode(s) may communicate with processor 50 of the IMD via a sensing lead(s) and a stimulating lead(s) connected or coupled to the IMD.

Although the foregoing invention has been described in some detail by way of illustration and example for purposes of clarity of understanding, certain changes and modifications may be practiced within the scope of the appended claims.

What is claimed is:

1. A method comprising:
positioning an electrical sensor within vasculature of a subject; and
directly sensing, via the electrical sensor during spontaneous respiration of the subject, efferent activity of a phrenic nerve of the subject.

2. The method of claim 1, wherein the electrical sensor comprises a sensing electrode.

3. The method of claim 2, wherein the sensing electrode is a component of a sensing lead.

4. The method of claim 1, wherein the electrical sensor is spaced from the heart of the subject in a cranial direction.

5. The method of claim 1, wherein the electrical sensor directly senses efferent activity of the right phrenic nerve of the subject.

6. The method of claim 5, wherein the electrical sensor is positioned within the superior vena cava, within the right subclavian vein, or at a junction between the superior vena cava and the right subclavian vein of the subject.

7. The method of claim 5, wherein the electrical sensor is positioned within a thoracic cavity adjacent to the phrenic nerve or at an extravascular location along a nerve course in a neck of the subject.

8. The method of claim 5, further comprising electrically stimulating the left phrenic nerve of the subject based on the sensed efferent activity of the right phrenic nerve.

9. The method of claim 8, wherein the left phrenic nerve is electrically stimulated by a stimulating electrode positioned within vasculature of the subject.

10. The method of claim 8, wherein the left phrenic nerve of the subject is stimulated in a pattern corresponding to a pattern of the efferent activity of the right phrenic nerve of the subject.

11. The method of claim 8, wherein a left side of the diaphragm of the subject is defective, and wherein the electrical stimulation of the left phrenic nerve affects movement of the left side of the diaphragm in a pattern that corresponds to a pattern of movement of a right side of the diaphragm of the subject.

12. The method of claim 1, wherein the electrical sensor directly senses efferent activity of the left phrenic nerve of the subject.

13. The method of claim 12, wherein the electrical sensor is positioned within an extravascular location along a left side of a neck, within a left pulmonary artery, within a left pericardiocophrenic vein, or a left thoracic cavity in the subject.

14. The method of claim 12, further comprising electrically stimulating the right phrenic nerve of the subject based on the sensed efferent activity of the left phrenic nerve.

15. The method of claim 14, wherein the right phrenic nerve is electrically stimulated by a stimulating electrode positioned within vasculature of the subject.

16. The method of claim 14, wherein the right phrenic nerve of the subject is stimulated in a pattern corresponding to a pattern of the efferent activity of the left phrenic nerve of the subject.

17. The method of claim 14, wherein a right side of the diaphragm of the subject is defective, and wherein the electrical stimulation of the right phrenic nerve affects movement of the right side of the diaphragm in a pattern that corresponds to a pattern of movement of a left side of the diaphragm of the subject.

18. The method of claim 1, wherein the electrical sensor is a component of, or in communication with, an implantable medical device.

19. A system comprising:
a. an electrical sensor configured to be positioned within vasculature of the subject and to sense efferent activity of one of the left phrenic nerve or the right phrenic nerve of the subject;
b. a stimulating electrode configured to be positioned within vasculature of the subject and to electrically stimulate the other of the left phrenic nerve or the right phrenic nerve of the subject;
c. a processor in communication with the electrical sensor and the stimulating electrode, wherein the processor is configured to receive signals from the electrical sensor corresponding to the sensed efferent activity of said one of the left phrenic nerve or the right phrenic nerve of the subject, and wherein the processor is further configured to cause the stimulating electrode to apply electrical stimulation to said other of the left phrenic nerve or the right phrenic nerve of the subject, wherein the electrical stimulation is configured to synchronize movement of left and right sides of the diaphragm of the subject.

20. The system of claim 19, wherein the electrical sensor comprises a sensing electrode.
